(19) United States
(12) Patent Application Publication
(10) Pub. No.: US 2013/0149199 A1
(43) Pub. Date: Jun. 13, 2013

(54) PITOT TUBE CONNECTION

(71) Applicant: Tenneco Automotive Operating Company Inc., Lake Forest, IL (US)

(72) Inventors: Jason Porter, Jackson, MI (US); Bradley Walworth, Jackson, MI (US); David French, Saline, MI (US); Kenneth Zebarah, Clark Lake, MI (US); Thomas Harris, JR., Jackson, MI (US)

(73) Assignee: TENNECO AUTOMOTIVE OPERATING COMPANY INC., Lake Forest, IL (US)

(21) Appl. No.: 13/706,461

(22) Filed: Dec. 6, 2012

Related U.S. Application Data
(60) Provisional application No. 61/568,222, filed on Dec. 8, 2011.

(51) Int. Cl. G01N 33/00

(52) U.S. Cl. CPC G01N 33/0004
USPC 422/119

(57) ABSTRACT

An exhaust treatment system includes an exhaust treatment device. The exhaust treatment device includes a shell, and a sensor boss configured to support a sensor device is mounted to the shell. A Pitot tube that is configured to communicate exhaust to the sensor device is coupled to the sensor boss at a proximate end thereof, while a distal end is affixed to the shell of the exhaust treatment device to prevent detachment of the Pitot tube from the sensor boss, and ensure that exhaust gases are effectively communicated to the sensor device.

### CROSS-REFERENCE TO RELATED APPLICATIONS

The present disclosure claims priority to U.S. Provisional Application Ser. No. 61/568,222, filed Dec. 8, 2011, the disclosure of which is hereby incorporated by reference in its entirety.

### FIELD

The present disclosure relates to an exhaust system including an exhaust treatment device.

### BACKGROUND

This section provides background information related to the present disclosure which is not necessarily prior art.

Exhaust gas sensors have been used in vehicles to sense the presence of constituents (e.g., oxygen, hydrocarbons, nitrous oxides, etc.) in an exhaust gas stream and to sense and/or signal, for example, when an internal combustion engine switches from rich to lean or from lean to rich operation, or when a catalyst of the exhaust treatment device is no longer operating within a desired range.

Because exhaust gas sensors are required to be positioned within an exhaust gas flow, the exhaust gas sensor is usually mounted to a portion of the exhaust system. Due to application requirements, the exhaust system itself may have unique exterior configurations, which may not be optimal for mounting exhaust sensors to the system (e.g., due to irregular mounting surfaces). Accordingly, exhaust system designs limit the positioning and configuration of the gas sensor within the exhaust system. To account for these drawbacks, a prefabricated mounting boss can be secured to the exhaust system by an attachment method wherein the boss is inserted into or about a hole pierced in the shell of the exhaust system component, and the boss is welded to the shell. Such a configuration, however, does not ensure that a sufficient amount of exhaust gases reach the sensor device.

In addition, the exhaust treatment device will typically have insulated catalyst bricks that are disposed within the shell. The catalyst bricks are wrapped in an insulating blanket (mat) disposed between the exterior of the catalyst brick and the interior surface of the exhaust treatment device. It is desirable to monitor the gases flowing through the catalyst bricks and to monitor the gas composition between the bricks. However, accurate sensing by known methods may require a sufficient amount of free space between the bricks for the sensing element of the gas sensor to be mounted within the flow path of and between the catalyst bricks (e.g., mid-stream mounting). Accordingly, it may be beneficial to provide an improved exhaust gas sensor system.

### SUMMARY

This section provides a general summary of the disclosure, and is not a comprehensive disclosure of its full scope or all of its features.

The present disclosure provides an exhaust treatment system including an exhaust treatment device including a shell; a sensor boss configured to support a sensor device mounted to the shell; and a communication tube configured to communicate exhaust to the sensor device. The communication tube includes a first end coupled to the sensor boss and a second end fixed to the shell.

Additional aspects of the exhaust treatment system include that the communication tube may be a Pitot tube, the first end may be press-fit to the sensor boss, and the second end may be welded to the shell.

Additionally, at least one catalytic brick may be disposed in the shell, wherein the communication tube is located downstream from the brick. Alternatively, the communication tube may be located upstream from the brick.

The communication tube may include at least one through hole for communicating exhaust to the sensor device. The communication tube may also include at least one exit aperture to allow gas to pass through the at least one through hole, communicate with the sensor device, and exit the communication tube. Preferably, the through hole faces an inlet of the exhaust treatment device.

Lastly, the exhaust system of the present disclosure can include a sensor boss that may include an asymmetrical feature that aligns the through hole toward an inlet of the exhaust treatment device.

The present disclosure also provides an exhaust treatment device including a shell; a catalyst brick disposed in the shell for treating an exhaust gas passing through the shell; a sensor in communication with the exhaust gas in the shell; a mounting structure for securing the sensor to the shell; and an exhaust gas communication tube disposed in the shell for directing at least a portion of the exhaust gas to the sensor, wherein a first end of the exhaust gas communication tube is non-fixedly coupled to the mounting structure, and a second opposite end of the exhaust gas communication tube is fixedly coupled to the shell.

The exhaust gas communication tube may include at least one inlet aperture for receiving and directing the portion of the exhaust gas to the sensor. Further, the exhaust gas communication tube may include at least one exit aperture for allowing the portion of the exhaust gas received and directed to the sensor by the inlet aperture to exit the gas communication tube.

The first end of the gas communication tube may be press-fit to the mounting structure, and the second end may be welded to the shell.

The catalyst brick of the exhaust treatment device may be selected from the group consisting of diesel oxidation catalysts, selective catalytic reduction catalysts, and catalyst-coated particulate filters.

The gas communication tube may be located downstream from the brick. Alternatively, the gas communication tube may be located upstream from the brick. Regardless, the at least one inlet aperture may face an inlet of the shell. To ensure that the at least one inlet aperture faces the inlet of the shell, the mounting structure may include an asymmetrical feature that aligns the at least one inlet aperture toward the inlet of the shell.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a schematic representation of an exhaust system according to a principle of the present disclosure;

FIG. 2 is a side-perspective view of an exhaust system treatment component according to a principle of the present disclosure;

FIG. 3 is a top-perspective view of the exhaust system treatment component illustrated in FIG. 2;

FIG. 4 is a cross-sectional view of the exhaust system treatment component illustrated in FIG. 2;

FIG. 4A is an enlarged view of a portion of FIG. 4; and

FIG. 5 is a cross-sectional view of the exhaust treatment component along line 5-5 illustrated in FIG. 4.

Corresponding reference numerals indicate corresponding parts throughout the several views of the drawings.

### DETAILED DESCRIPTION

Example embodiments will now be described more fully with reference to the accompanying drawings.

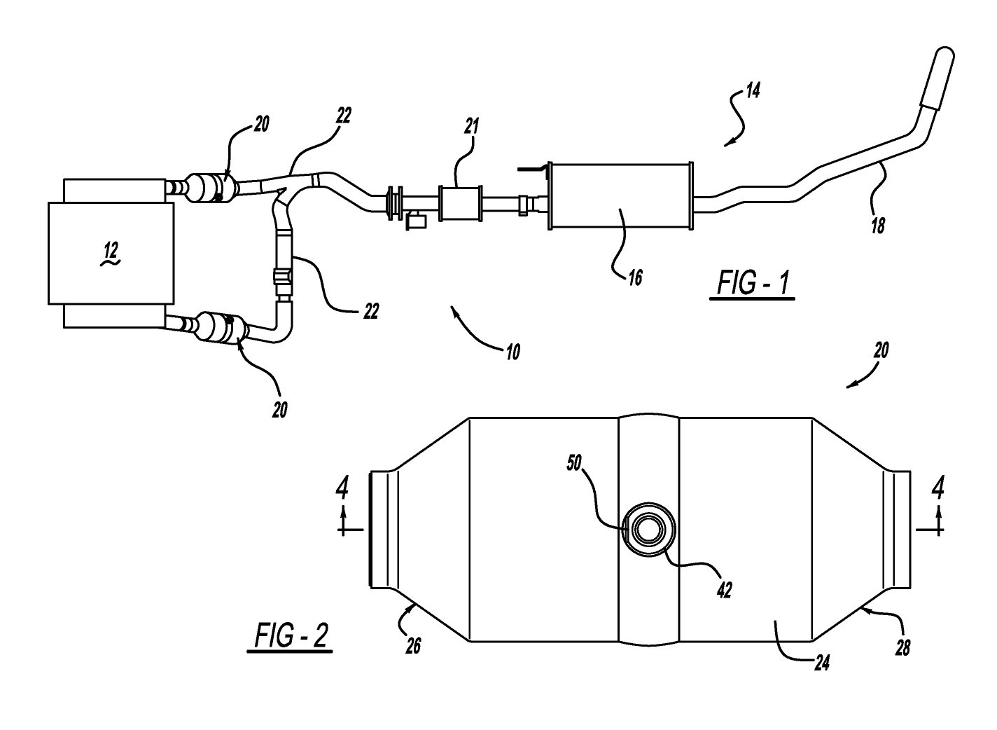

FIG. 1 schematically illustrates an exhaust system 10 according to the present disclosure. Exhaust system 10 includes at least an internal combustion engine 12 and a tailpipe assembly 14 including, for example, a muffler 16 and exhaust outlet pipe 18. Engine 12 can be any type of internal combustion engine known to one skilled in the art such as, for example, a gasoline engine or a diesel engine. Exhaust system 10 may also include at least one exhaust treatment device 20 disposed downstream from engine 12 and upstream from tailpipe assembly 14. In the exemplary illustrated embodiment, exhaust system 10 may include a pair of exhaust treatment devices 20 connected via exhaust lines 22 to tail pipe assembly 14. Between exhaust treatment devices 20 and tailpipe assembly 14 may be disposed an extension pipe assembly 21.

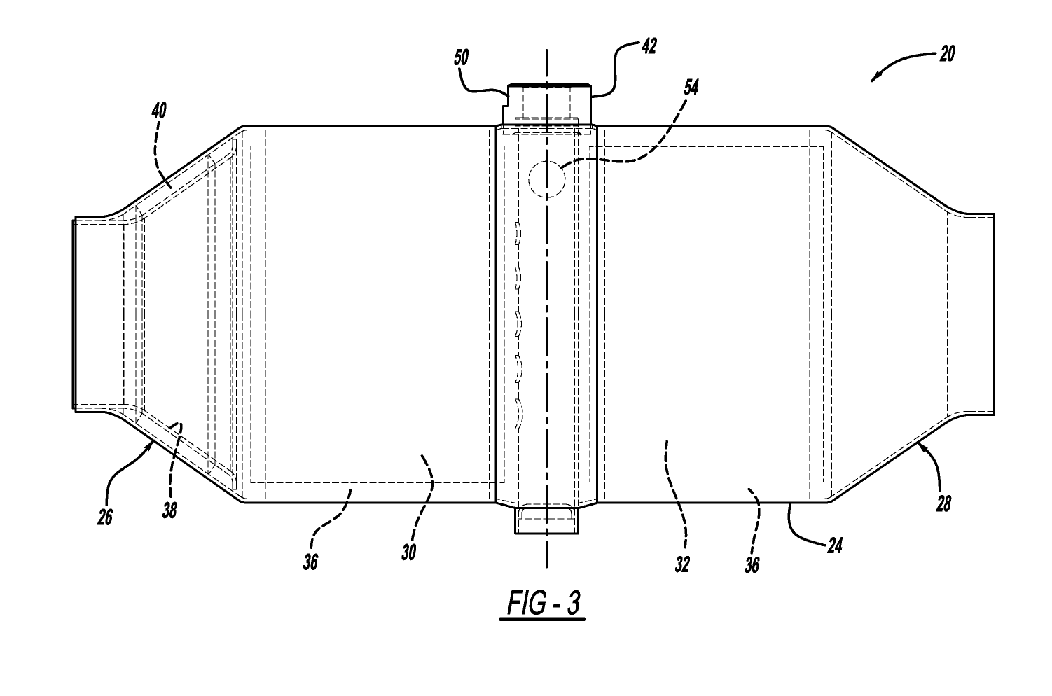

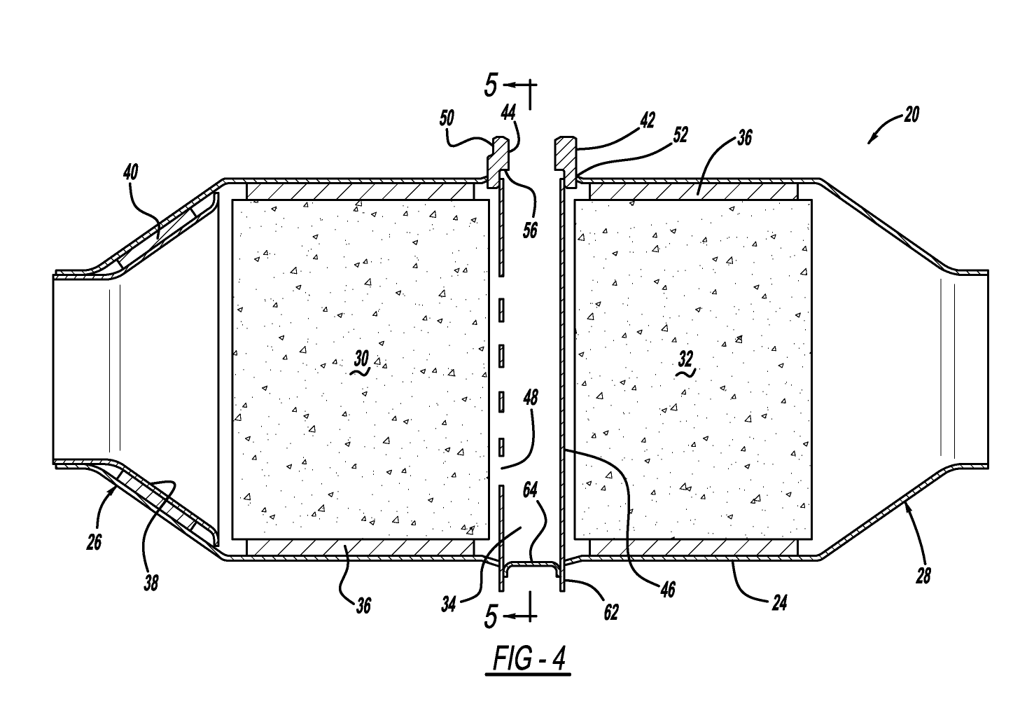

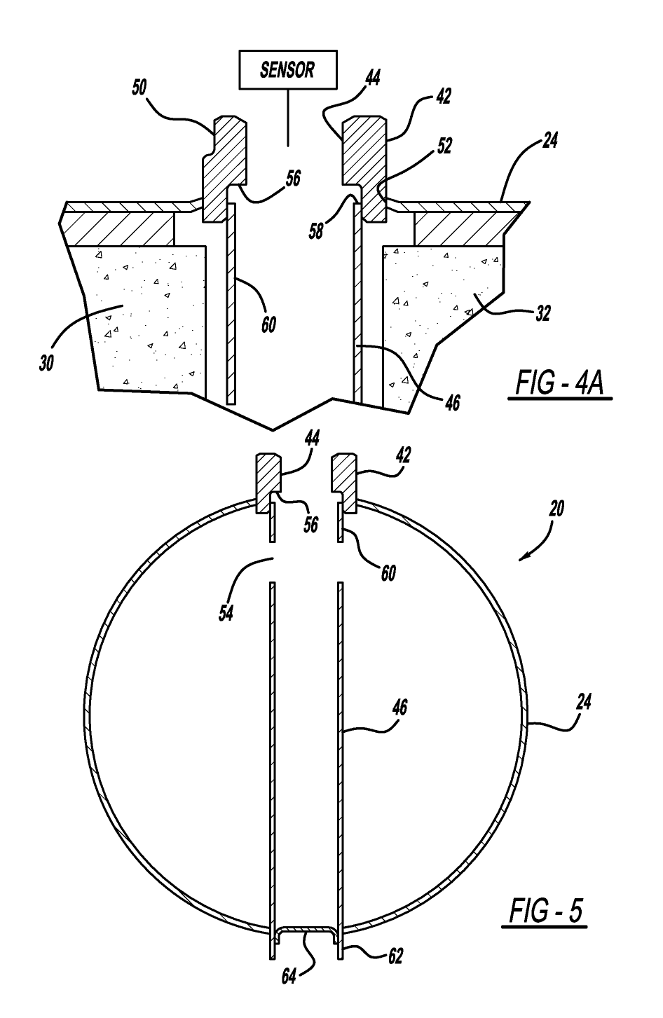

Now referring to FIGS. 2-6, a configuration of exhaust treatment device 20 will be described. Exhaust treatment device 20 may include an outer shell 24 having an inlet end 26 and an outlet end 28. Proximate to inlet end 26 is a first catalyst brick 30 and spaced therefrom proximate outlet end 28 is a second catalyst brick 32. First and second catalyst bricks 30 and 32 may be spaced apart by gap 34, and may be any type of catalyst brick known in the art. In some exemplary embodiments, first and second catalyst bricks 30 and 32 may be gas converter bricks. In other exemplary embodiments, first and second catalyst bricks 30 and 32 may be selective catalytic reduction (SCR) catalyst, diesel oxidation catalyst (DOC), catalyst-coated diesel particulate filter (DPF) bricks, or combinations thereof. Regardless, disposed between outer shell 24 and each catalyst brick 30 and 32 may be an insulating mat 36.

Although inlet end 26 and outlet end 28 are illustrated as being integral or unitary with outer shell 24, it should be understood that inlet end 26 and outlet end 28 may be separately formed pieces that are subsequently welded to outer shell 24. In this regard, in some exemplary embodiments, outer shell 24, inlet end 26, and outlet end 28 are pieces separately formed of materials such as stainless steel. Other materials, such as, for example, aluminum, are contemplated however. Inlet end 26 may also include an inner shell 38 coupled thereto. Between inner shell 38 and inlet end 26 can be formed another insulating mat 40. Inlet end 26 may include inner shell 38 and insulating mat 40 due to higher exhaust temperatures generally being present at inlet end 26 in comparison to outlet end 28. It should be understood, however, that outlet end 28 may also be formed to include an inner shell and insulating mat without departing from the scope of the present disclosure.

During use of exhaust treatment device 20, it is often desirable to use sensor devices to take various measurements as exhaust gases travel through exhaust treatment device. For example, in some embodiments, sensor devices such as a pressure sensor, a CMS sensor, a NOₓ sensor, an oxygen (O₂) sensor, an ammonia (NH₃) sensor, a particulate matter sensor, a temperature sensor, or any other type of sensor known to one skilled in the art of exhaust treatment may be used to monitor the exhaust gas in order to better determine what operating parameters of the exhaust treatment system need to be adjusted.

To connect the sensor device to exhaust treatment device 20, a sensor boss 42 may be attached to exhaust treatment device 20. As illustrated in FIGS. 2 to 5, sensor boss 42 is attached to outer shell 24 of exhaust treatment device 20 at a position located between catalyst bricks 30 and 32. It should be understood, however, that sensor boss 42 is not limited to being attached to outer shell 24 at a position located between catalyst bricks 30 and 32. Rather, the present disclosure contemplates that sensor boss 42 may be located upstream of catalyst brick 30 (i.e., adjacent inlet 26), or downstream of catalyst brick 32 (i.e., adjacent outlet 28). Regardless, sensor boss 42 may be formed of materials such as stainless steel, aluminum, or other metal materials that facilitate welding or brazing to outer shell 24 such that sensor boss 42 is rigidly attached to outer shell 24. To facilitate communication with the exhaust gases to be monitored by the sensor device, sensor boss 42 has a centrally disposed aperture 44 that provides communication between the sensor device and the exhaust gases passing through exhaust treatment device 20. To connect the sensor to the sensor boss 42, a portion of aperture 44 may be threaded (not shown). Other connection methods between the sensor device and the sensor boss 42, however, may be used.

Aperture 44 may not be sufficiently sized or positioned to effectively communicate exhaust gases passing through exhaust treatment device 20 to the sensor device. To ensure that a sufficient portion of the exhaust gases are communicated through aperture 44 to the sensor device, an exhaust gas communication tube 46 (hereinafter “Pitot tube”) may be used. Pitot tube 46 may be a generally cylindrical tube formed of a material such as stainless steel, aluminum, titanium, or any other material satisfactory for withstanding exposure to the exhaust gases. To satisfactorily facilitate entry of the exhaust gases into Pitot tube 46 to the sensor device, Pitot tube 46 includes at least one through hole 48.

To ensure that through holes 48 are directed toward inlet end 26 to receive a flow of the exhaust gases, sensor boss 42 includes an asymmetrical feature (stepped portion) 50. Pitot tube 46 may be press-fit or otherwise secured to sensor boss 42. As Pitot tube 46 is secured to sensor boss 42, through holes 48 may be aligned to face in the same direction as stepped portion 50. Then, when sensor boss 42 including the press-fit Pitot tube 46 is placed through a first opening 52 in shell 24, sensor boss 42 may be manipulated by the assembler such that stepped portion 50 faces toward inlet end 26. As through holes 48 are aligned in the same direction as stepped portion 50, when stepped portion 50 is aligned to face toward inlet end 26, through holes 48 are also aligned to face inlet end 26. Exhaust gases passing through exhaust treatment device 20 are then ensured to pass into through holes 48 and be in communication with the sensor device. After properly aligning sensor boss 42 and Pitot tube 46, sensor boss 42 can be secured to shell 24 by welding or brazing. Exemplary welding techniques include metal inert gas (MIG) welding techniques, but other welding techniques may be used without departing from the scope of the present disclosure.

To provide an exit for exhaust gases that communicate with the sensor device, Pitot tube 46 may also include secondary through holes 54. Secondary through holes 54 may be arranged orthogonal to through holes 48 to ensure that the exhaust gases entering through holes 48 do not simply pass through Pitot tube 46 without at least a portion of the exhaust gases first being communicated to the sensor device.

In the embodiment where Pitot tube 46 is press-fit to sensor boss 42, central aperture 44 is radially expanded to provide a shoulder 56. Shoulder 56 may be spaced apart from a terminal end 58 of Pitot tube 46 to provide for thermal expansion of Pitot tube 46 during use of exhaust treatment device 20. In this regard, during use of the exhaust treatment device 20, the exhaust gases will be high temperature, which will cause Pitot tube 46 to expand during use of engine 12. When engine 12 is not operating, Pitot tube 46 will cool, which will cause Pitot tube 46 to contract to its original size.

Press-fitting Pitot tube 46 to sensor boss 42 allows for Pitot tube 46 to expand and contract without detaching from sensor boss 42 during use of exhaust treatment device 20. This is a beneficial aspect of the present disclosure because if Pitot tube 46 was rigidly fixed to sensor boss 42, the expansion and contraction of Pitot tube 46 can weaken the connection between Pitot tube 46 and sensor boss 42, which increases the risk of Pitot tube 46 eventually detaching from sensor boss 42. In such a case, the efficacy of the sensor can be reduced because the amount of exhaust gases reaching the sensor will be reduced without the aid of Pitot tube 46.

To further ensure that Pitot tube 46 does not detach from sensor boss 42, Pitot tube 46 has a length that allows Pitot tube 46 to extend entirely through shell 24. That is, a first end 60 may be press-fit to sensor boss 42, while a second end 62 extends through shell 24. Second end 62, in some exemplary embodiments, may be welded or brazed to shell 24. To ensure that exhaust treatment device 20 remains gas tight, a cap 64 may be fixed to second end 58 of Pitot tube 48. Cap 64 may be fixed to second end 62 by welding, brazing, or any other attachment method known to one skilled in the art so long as exhaust treatment device 20 remains sealed.

According to one arrangement discussed above, Pitot tube 46 is press-fit to sensor boss 42 to account for thermal expansion, and rigidly attached to shell 24. In this manner, Pitot tube 46 is prevented from detaching from sensor boss 42 during use of exhaust treatment device 20, which ensures that a satisfactory amount of exhaust gases are communicated to the sensor device.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. An exhaust treatment system, comprising
an exhaust treatment device including a shell;
a sensor boss configured to support a sensor device mounted to said shell; and
a communication tube configured to communicate exhaust to the sensor device,
wherein said communication tube includes a first end coupled to said sensor boss and a second end fixed to said shell.

2. The exhaust treatment system of claim 1, wherein said communication tube is a Pitot tube.

3. The exhaust treatment system of claim 1, wherein said first end is press-fit to said sensor boss.

4. The exhaust treatment system of claim 1, wherein said second end is welded to said shell.

5. The exhaust treatment system of claim 1, further comprising at least one catalytic brick disposed in said shell.

6. The exhaust treatment system of claim 5, wherein said communication tube is located downstream from said brick.

7. The exhaust treatment system of claim 5, wherein said communication tube is located upstream from said brick.

8. The exhaust treatment system of claim 1, wherein said communication tube includes at least one through hole for communicating exhaust to said sensor device.

9. The exhaust treatment system of claim 8, wherein said communication tube includes at least one exit aperture to allow gas to pass through the at least one through hole, communicate with the sensor device, and exit the communication tube.

10. The exhaust treatment system of claim 8, wherein said through hole faces an inlet of said exhaust treatment device.

11. The exhaust treatment system of claim 8, wherein said sensor boss includes an asymmetrical feature that aligns said through hole toward an inlet of said exhaust treatment device.

12. An exhaust treatment device, comprising:
a shell;
a catalyst brick disposed in the shell for treating an exhaust gas passing through the shell;
a sensor in communication with the exhaust gas in the shell;
a mounting structure for securing the sensor to the shell; and
an exhaust gas communication tube disposed in the shell for directing at least a portion of the exhaust gas to the sensor,
wherein a first end of the exhaust gas communication tube is non-fixedly coupled to mounting structure, and a second opposite end of the exhaust gas communication tube is fixedly coupled to the shell.

13. The exhaust treatment device of claim 12, wherein the exhaust gas communication tube includes at least one inlet aperture for receiving and directing the portion of the exhaust gas to the sensor.

14. The exhaust treatment device of claim 13, wherein the exhaust gas communication tube includes at least one exit aperture for allowing the portion of the exhaust gas received and directed to the sensor by the inlet aperture to exit the gas communication tube.

15. The exhaust treatment device of claim 12, wherein the first end is press-fit to the mounting structure.

16. The exhaust treatment device of claim 12, wherein the second end is welded to the shell.

17. The exhaust treatment device of claim 12, wherein the catalyst brick is selected from the group consisting of diesel oxidation catalysts, selective catalytic reduction catalysts, and catalyst-coated particulate filters.

18. The exhaust treatment device of claim 12, wherein the tube is located downstream from the brick.

19. The exhaust treatment device of claim 12, wherein said tube is located upstream from said brick.

20. The exhaust treatment device of claim 13, wherein the at least one inlet aperture faces an inlet of the shell.

21. The exhaust treatment device of claim 20, wherein the mounting structure includes an asymmetrical feature that aligns the at least one inlet aperture toward the inlet of the shell.
